(19) United States
(12) Patent Application Publication
(10) Pub. No.: US 2020/0123484 A1
(43) Pub. Date: Apr. 23, 2020

(54) INTEGRATED CHIP AND METHOD FOR SPERM SORTING, OOCYTE INCUBATION, AND IN VITRO FERTILIZATION

(71) Applicant: National Tsing Hua University, Hsinchu City (TW)

(72) Inventors: Fan-Gang Tseng, Taipei City (TW); Yung-Chin Tzeng, Hsinchu City (TW); Yueh-Jen Chen, Hsinchu City (TW); Chuan Chang, Hsinchu City (TW)

(73) Assignee: National Tsing Hua University, Hsinchu City (TW)

(21) Appl. No.: 16/166,167

(22) Filed: Oct. 22, 2018

(51) Int. Cl. C12M 3/00; C12N 5/071; C12N 5/073; C12N 5/075; C12M 3/06; C12M 1/00

(52) U.S. Cl. CPC C12M 21/06; C12N 5/0612; C12M 29/10; C12N 5/0609; C12M 23/16; C12N 5/0604

(57) ABSTRACT

An integrated chip is provided and comprises systems for sperm sorting, oocyte incubation and fertilization and liquid perfusion. The sperm sorting system comprises an inlet, an outlet, a main channel and a branch channel. The main channel has a width which increases along a direction from the inlet toward the outlet. The branch channel is connected to the main channel. The oocyte incubation and fertilization system is connected to the branch channel and comprises upper and lower holes. The upper hole is larger than the oocyte to hold the oocyte to be fertilized by a sperm. The lower hole is smaller than the oocyte. The sorted sperms are introduced into the oocyte incubation and fertilization system through the branch channel, to fertilize with the oocytes in the upper holes. The liquid perfusion system is located below the lower holes and a liquid exchange is performed through the second holes.

### BACKGROUND

### Technical Field

The invention relates to a system and a method, and more particularly, to an integrated chip and a method for sperm sorting, oocyte incubation, and in vitro fertilization.

### Description of Related Art

According to statistical data, the proportion of infertile women at child-bearing age in Taiwan is about 10 to 15. Facing with such a high infertility rate, the rate of successful in vitro fertilization (IVF) (also known as “test tube baby”) was only 33%. Such low success rate is mostly attributed to the facts that during the traditional IVF techniques, the treatment methods of specimens are not moderate enough or human errors result in a significant decline in the health or even death of the specimens. If a system may be developed to handle the specimens in a more gentle method. It is believed that the success rate of in vitro fertilization may be significantly improved.

### SUMMARY

The invention provides an integrated chip for sperm sorting, oocyte incubation, and in vitro fertilization.

The invention further provides a method for sperm sorting, oocyte incubation, and in vitro fertilization.

The invention provides an integrated chip for sperm sorting, oocyte incubation, and in vitro fertilization, and the integrated chip includes a sperm sorting system, an oocyte incubation and fertilization system, and a liquid perfusion system. The sperm sorting system includes a sperm inlet, a sperm outlet, a main channel, and a branch channel. The main channel is connected to the sperm inlet and the sperm outlet and has a width which gradually increases along a direction from the sperm inlet toward the sperm outlet. The branch channel is located between the sperm inlet and the sperm outlet and is connected to the main channel. The oocyte incubation and fertilization system is connected to the branch channel, and includes a first hole part and a second hole part. The first hole part includes a plurality of first holes, and a diameter of the first hole is greater than an average diameter of an oocyte for holding the oocyte to be fertilized by a sperm. The second hole part is located below the first hole part and includes a plurality of second holes, and a diameter of the second hole is smaller than the oocyte for liquid exchange. The sperm sorted by the sperm sorting system is introduced into the oocyte incubation and fertilization system through the branch channel to fertilize with the oocyte located in the first hole. The liquid perfusion system includes a liquid inlet, a liquid outlet, and a liquid channel. The liquid channel is located between and connected to the liquid inlet and the liquid outlet. A portion of the liquid channel is located below the second hole part, and liquid is exchanged between the oocyte incubation and fertilization system and the liquid channel through the second hole part.

In an embodiment of the invention, a material of the first hole part and the second hole part includes polydimethylsiloxane (PDMS).

In an embodiment of the invention, the first holes are arranged regularly.

In an embodiment of the invention, the second holes are arranged regularly.

In an embodiment of the invention, the diameter of the second hole is greater than 30 urn.

In an embodiment of the invention, the main channel includes a first section and a second section connected to the first section. The first section is located between the sperm inlet and the second section, and the second section is located between the first section and the sperm outlet. The first section has a substantially identical width, the second section has a gradually increasing width, and a minimum width of the second section is greater than the width of the first section.

In an embodiment of the invention, a width of the branch channel is smaller than the minimum width of the second section.

In an embodiment of the invention, the sperm sorting system further includes an obstruction structure. The obstruction structure is located in the main channel and adjacent to the sperm outlet, and a distance is between the obstruction structure and a sidewall of the main channel.

In an embodiment of the invention, the obstruction structure is dumbbell-shaped.

In an embodiment of the invention, the sperm inlet and the sperm outlet are tanks respectively.

In an embodiment of the invention, the liquid channel has a liquid exchange region located below the oocyte incubation and fertilization system and having a contour consistent with the second hole part.

In an embodiment of the invention, the integrated chip includes a first film, a second film located below the first film, a third film located below the second film, and a fourth film located below the third film. The first film includes a first through hole corresponding to the sperm inlet, a second through hole corresponding to the sperm outlet, a first trench corresponding to the main channel and the branch channel, a third through hole corresponding to the oocyte incubation and fertilization system, a fourth through hole corresponding to the liquid inlet, and a fifth through hole corresponding to the liquid outlet. The second film includes the first hole part, a sixth through hole corresponding to the fourth through hole, and a seventh through hole corresponding to the fifth through hole. The second film located below the first through hole, the second through hole, and the first trench are respectively served as the sperm inlet, the sperm outlet, and bottoms of the main channel and the branch channel. The third film includes the second hole part, an eighth through hole corresponding to the sixth through hole, and a ninth through hole corresponding to the seventh through hole. The fourth film includes a first groove, a second trench, and a second groove which are connected. The first groove corresponds to the eighth through hole and serves as a bottom of the liquid inlet, the second trench serves as the liquid channel, the second groove corresponds to the ninth through hole and serves as a bottom of the liquid outlet.

In an embodiment of the invention, a material of the first film, the second film, the third film, and the fourth film includes polydimethylsiloxane.

In an embodiment of the invention, the adjacent two of the first film, the second film, the third film, and the fourth film are adhered to each other.

The invention provides a method for sperm sorting, oocyte incubation, and in vitro fertilization and the method includes the following steps. The aforementioned integrated chip is provided. A sperm-containing sample is dropped into the sperm inlet, so that a sperm sorting is performed on the sperm-containing sample by the sperm sorting system. An oocyte-containing sample is dropped into the oocyte incubation and fertilization system, so that the oocytes is located in the first hole respectively. The sorted sperm is introduced into the oocyte incubation and fertilization system through the branch channel, so that the sorted sperm and the oocyte are fertilized to form a fertilized egg in the first hole.

In an embodiment of the invention, the method further includes performing a zona pellucida removal step on the oocyte after the oocyte-containing sample is dropped into the oocyte incubation and fertilization system.

In an embodiment of the invention, the method further includes: after the oocyte-containing sample is dropped into the oocyte incubation and fertilization system, sealing the oocyte incubation and fertilization system; and after the sperm sorting of the sperm-containing sample is finished by the sperm sorting system, opening the oocyte incubation and fertilization system so that the sorted sperm enters the oocyte incubation and fertilization system through the branch channel.

In an embodiment of the invention, the method further includes: after the sperm-containing sample is dropped into the sperm inlet, sealing the sperm sorting system; and after the oocyte-containing sample is dropped into the oocyte incubation and fertilization system, opening the sperm sorting system to perform the sperm sorting.

In an embodiment of the invention, the method further includes culturing the fertilized egg in the oocyte incubation and fertilization system.

In an embodiment of the invention, the method further includes flowing liquid in the liquid perfusion system, wherein the liquid in the liquid perfusion system is exchanged with liquid in the oocyte incubation and fertilization system through the second hole part.

Based on the above, the invention integrates the sperm sorting system, the oocyte incubation and fertilization system, and the liquid perfusion system onto a single chip, in which high-efficiency sperm sorted by the sperm sorting system may be directly introduced into the oocyte incubation and fertilization system, and the high-efficiency sperm and the cultivated oocyte may be fertilized in situ to form a fertilized egg. Accordingly, in vitro fertilization is achieved. Since sperm sorting, treatment on oocyte, and fertilized egg formation are passively performed on the chip, the processes thereof are gentle and harm to sperm and oocyte is avoid, and survival rate and fertilization rate are significantly increased. As a result, the chip of the invention may not only shorten the operation time of the traditional artificial reproduction technique, it also simplifies the operation process to avoid the death of the specimen due to the long operation time and the human-induced misoperation, thereby greatly improving the success rate of the artificial reproductive technique.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1A is a perspective schematic diagram of an integrated chip according to an embodiment of an invention, FIG. 1B is an exploded schematic diagram of the integrated chip of FIG. 1A, FIG. 1C is a bottom view of the integrated chip of FIG. 1A, and FIG. 1D is a cross sectional diagram of the integrated chip of FIG. 1A.

### DESCRIPTION OF THE EMBODIMENTS

The following disclosure provides many different embodiments, or examples, for implementing different features of the provided subject matter. Specific examples of components and arrangements are described below to simplify the disclosure. These are, of course, merely examples and are not intended to be limiting. For example, the formation of a second feature over or over a first feature in the description that follows may include embodiments in which the second and first features are formed in direct contact, and may also include embodiments in which additional features may be formed between the second and first features, such that the second and first features may not be in direct contact. In addition, the disclosure may repeat reference numerals and/or letters in the various examples. This repetition is for the purpose of simplicity and clarity and does not in itself dictate a relationship between the various embodiments and/or configurations discussed.

The invention utilizes the nature of the sperm that swims against the flow and divergent flow field, so that by progressively reducing flow rate through increasing the cross-sectional area of a portion of the channel, the sperms are sorted based on motility. Specifically, the active sperm will remain in situ or swim against the flow by moving against fluid (i.e., fluid in the sample), while the less active sperm will be carrier to the outlet. Additionally, the invention also utilizes the obstruction structure to block the active sperm and accelerate the less active sperm or dead sperm to flow toward the outlet. More importantly, the branch channel is utilized in the invention, and the sorted sperm may be introduced into the oocyte incubation and fertilization system through the branch channel.

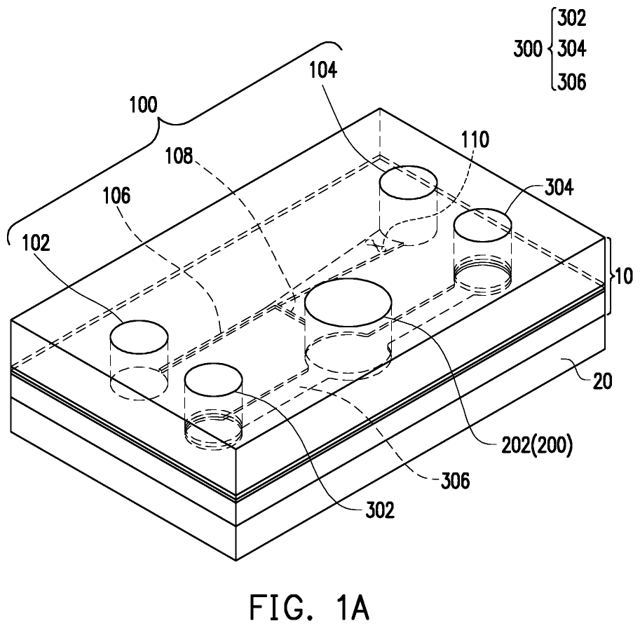

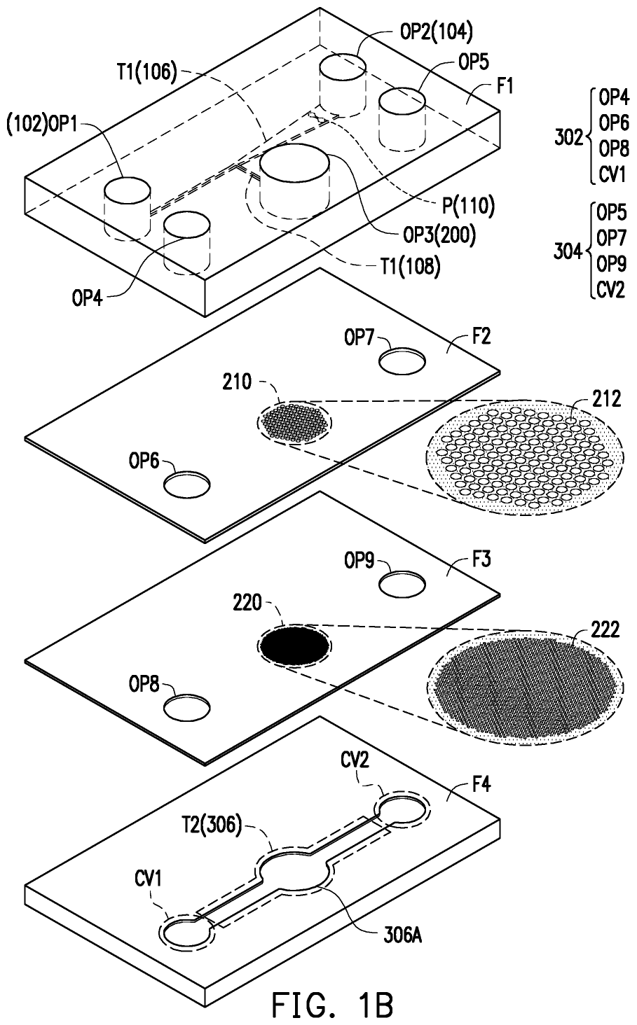

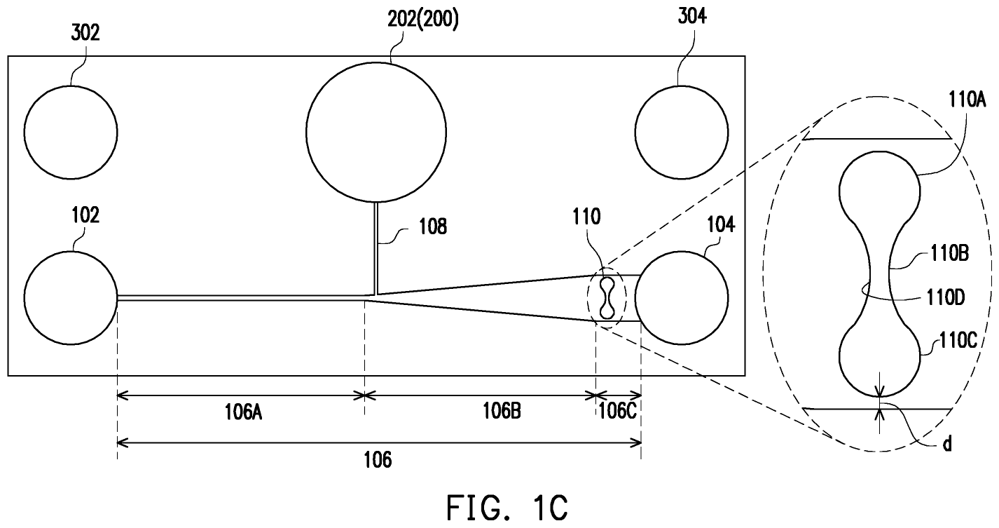

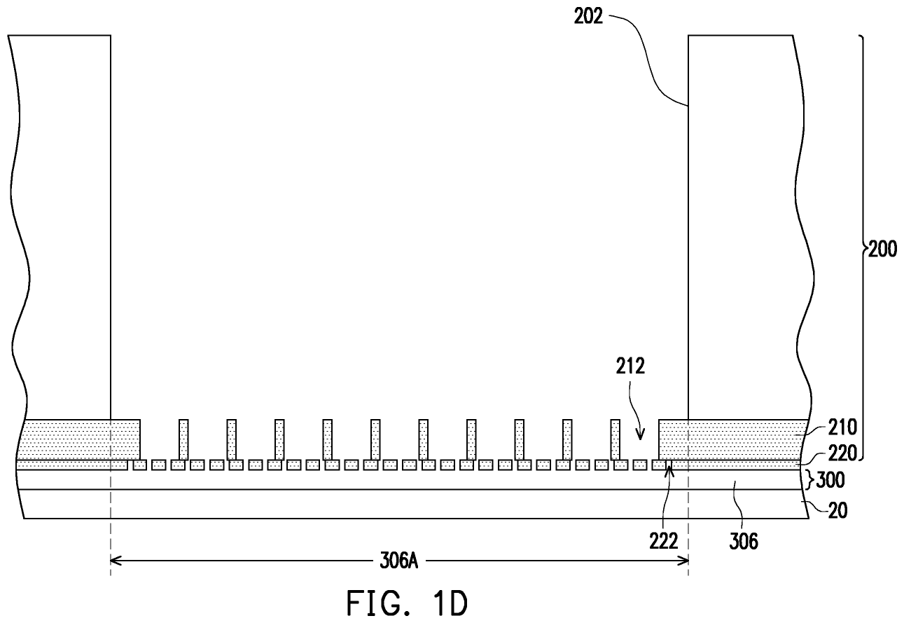

FIG. 1A is a perspective schematic diagram of an integrated chip according to an embodiment of an invention, FIG. 1B is an exploded schematic diagram of the integrated chip of FIG. 1A, FIG. 1C is a bottom view of the integrated chip of FIG. 1A, and FIG. 1D is a cross sectional diagram of the integrated chip of FIG. 1A. Referring also to FIGS. 1A to 1D, an integrated chip 10 includes a sperm sorting system 100, an oocyte incubation and fertilization system 200, and a liquid perfusion system 300.

The sperm sorting system 100 includes a sperm inlet 102, a sperm outlet 104, a main channel 106, and a branch channel 108. In some embodiments, the sperm inlet 102 is configured to receive a sperm-containing sample. The sperm outlet 104 is configured to receive liquid flowing out of the main channel 106. In some embodiments, the sperm inlet 102 and the sperm outlet 104 are respectively tanks, for example. The diameter of the sperm inlet 102 and the sperm outlet 104 is 1.8 mm to 2.2 mm, and is 2 mm, for example. The depth of the sperm inlet 102 and the sperm outlet 104 is 1.8 mm to 2.2 mm, and is 2 mm, for example. The main channel 106 and the branch channel 108 are respectively trenches having a depth, for example. The main channel 106 is located between and connected to the sperm inlet 102 and the sperm outlet 104.

In some embodiments, when the integrated chip 10 is applied, the sperm-containing sample is placed at the sperm inlet 102, and due to the difference in liquid height between the sperm inlet 102 and the main channel 106, the sample enters the main channel 106 from the sperm inlet 102. In the main channel 106, the active sperm in the sample will remain in situ or swim against the flow by moving against fluid while the less active sperm in the sample will be carrier to the sperm outlet 104.

In some embodiments, the main channel 106 includes a first section 106A, a second section 106B, and a third section 106C which are connected, and they are sections for classifying and sorting the sperms. The first section 106A is located between the sperm inlet 102 and the second section 106B and has a substantially identical width, and the width is less than or equal to the minimum width of the second section 106B. The second section 106B is located between the first section 106A and the third section 106C, and has a width that gradually increases along the direction from the sperm inlet 102 toward the sperm outlet 104. As a result, the second section 106B may also be referred to as a progressively enlarged section. Since the flow rate of fluid is inversely proportional to the width of the channel it passes through, by different flow rates creating through the progressively enlarged section, the sperms are sorted based on motility and stay in the corresponding section. Specifically, the vertical cross-sectional area of the progressively enlarged section gradually increases along the direction from the sperm inlet 102 toward the sperm outlet 104. The fluid flow rate created by this design may be calculated by the fluid mechanics formula. The active sperm moves against the fluid and stays at different locations corresponding to different fluid rate depending on the sperm activity. It should be noted that the aforementioned vertical cross section refers to the vertical cross section which is perpendicular to an extended length direction of the sperm sorting system 100. More specifically, if necessary, the vertical cross-sectional area may be optionally interpreted as the vertical cross-sectional area through which the fluid flows, and the area occupied by the material forming the sperm sorting system 100 should be ignored.

Furthermore, in order to block the high activity sperm, the third section 106C is additionally configured in the main channel 106 of the sperm sampling system 100, and the third section 106C is also called the obstruction region. The third section 106C is located between the second section 106B and the sperm outlet 104, and is a section adjacent to the sperm outlet 104 in the main channel 106. In some embodiments, the third section 106C has, for example, a substantially identical width and this width is greater than or equal to the maximum width of the second section 106B. In the third section 106C, the fluid flows at a flow rate that is, for example, three times less than the specified flow rate, except that it is not limited to three times, and the flow rate of the fluid in the third section 106C may be three times to twenty times less than the specified flow rate. In some embodiments, for example, an obstruction structure 110 is disposed in the third section 106C of the sperm sorting system 100, and the obstruction structure 110 is configured to reduce the width of the channel through which the fluid flows, thereby increasing the flow rate of the fluid flowing through the third section 106C. In some embodiments, the obstruction structure 110 is, for example, located at the junction of the second section 106B and the third section 106C. In some embodiments, the obstruction structure 110 is, for example, in the shape of a dumbbell, and the dumbbell structure of the obstruction structure 110 has, for example, a head 110A, a connection portion 110B, and an end 110C, and the connection portion 110B is connected to and located between the head 110A and the end 110C. In some embodiments, a distance d is formed between the head 110A and the side wall of the third section 106C and between the end 110C and the side wall of the third section 106C. The distance d is, for example, greater than or equal to 50 μm. The arrangement direction of the head 110A, the connection portion 110B, and the end 110C is, for example, perpendicular to the flow direction of the fluid, so that the fluid may be temperately blocked at the connection portion 110B of the obstruction structure 110 and then immediately flow beyond the obstruction structure 110.

In some embodiments, the head 110A, the connection portion 110B, and the end 110C of the obstruction structure 110 are, for example, collectively formed a sperm collection tank 110D for storing sperms. The sperm collection tank 110D has a depth, and a depth extending direction is, for example, the same as the flow direction of the fluid. Based on the design of the sperm collection tank 110D, it may effectively separate dead and live sperms. In some embodiments, the average vertical cross-sectional area of the main channel 106 between sidewalls of the third section 106C and the obstruction structure 110 is, for example, between one-third and one-twentieth of the average vertical cross-sectional area of the main channel 106 at the junction between the second section 106B and the third section 106C. Thus, the flow rate of the flow will be significantly increased, for example, between three and twenty times.

The branch channel 108 is in communication with the main channel 106 and is configured to connect the sperm sorting system 100 with the oocyte incubation and fertilization system 200. In some embodiments, the high-efficiency sperm sorted by the sperm sorting system 100 will swim against the flow to the junction of the branch channel 108 and the main channel 106 and be passively introduced into the branch channel 108, and then introduced into the oocyte incubation and in vitro fertilization system 200. In some embodiments, the branch channel 108 is, for example, connected to the front end of the second section 106B, that is, the region through which the sample enters into the second section 106B from the first section 106A. The extending direction of the branch channel 108 is, for example, perpendicular to the extending direction from the sperm inlet 102 to the sperm outlet 104.

In some embodiments, the branch channel 108 has, for example, a substantially identical width, and this width is smaller than the minimum width of the main channel 106. That is, the width is smaller than the width of the first section 106A. In some embodiments, the width of the branch channel 108 ranges from 40 μm to 60 μm, and is, for example, 50 μm, and the width of the first section 106A ranges from 80 μm to 120 μm, and is, for example, 100 μm. In some embodiments, the length of the branch channel 108 is 1.8 mm to 2.2 mm, and is, for example, 2 mm, and the length of the main channel 106 is 8 mm to 10 mm, and is, for example, 12 mm. In some embodiments, the depth of the main channel 106 and the branch channel 108 is, for example, the same, and ranges from 80 μm to 120 μm. In some embodiments, the depth of the main channel 106 and the branch channel 108 is, for example, 100 μm.

The oocyte incubation and fertilization system 200 includes a tank 202 and a first hole part 210 and a second hole part 220 located in the tank 202. The tank 202 is in communication with the branch channel 108. The diameter of the tank 202 is 2.5 mm to 3.5 mm, and is, for example, 3 mm, and the depth of the tank 202 is 1.8 mm to 2.2 mm, and is, for example, 2 mm. The first hole part 210 includes a plurality of first holes 212, and the diameter of the first hole 212 is greater than the average diameter of the oocyte for holding the oocyte to be fertilized by a sperm. That is, the sperm sorted by the sperm sorting system 100 is introduced into the oocyte incubation and fertilization system 200 through the branch channel 108 to fertilize with the oocyte located in the first hole 212. In some embodiments, the diameter of the first hole 212 is designed such that the first hole 212 substantially contains only one oocyte, which facilitates the effective formation of a fertilized egg and the subsequent tracking of a single target fertilized egg. In some embodiments, the first hole part 210 is, for example, made of polydimethylsiloxane (PDMS), the first hole 212 penetrates the polydimethylsiloxane. The diameter of the first hole 212 ranges from 180 μm to 220 μm, and is 200 μm, and the depth of the first hole 212 ranges from 180 μm to 220 μm, and is 200 μm, for example. The first holes 212 are arranged regularly, for example, and porosity of sphere packing ranges from 50% to 60%, for example.

The second hole part 220 is located below the first hole part 210, and the second hole part 220 includes a plurality of second holes 222. The diameter of the second hole 222 is smaller than the oocyte, so that the oocyte and the fertilized egg may be maintained in the first hole 212, and the second hole 222 may serve as the bottom of the tank 202. In addition, the second hole 222 is capable of performing liquid exchange with the liquid perfusion system 300 underneath due to a pressure difference, and thus it is also called a liquid exchange film. In some embodiments, the second hole part 220 is made, for example, of polydimethylsiloxane, the second hole 222 penetrates the polydimethylsiloxane. The diameter of the second hole 222 ranges from 25 μm to 35 μm, and is, for example, 30 μm, and the depth of the second hole 222 ranges from 50 μm to 100 μm, and is, for example, 75 μm. The second holes 222 are arranged regularly, and porosity of sphere packing ranges from 15% to 30%, for example.

The liquid perfusion system 300 includes a liquid inlet 302, a liquid outlet 304, and a liquid channel 306 disposed between and in communication with the liquid inlet 302 and the liquid outlet 304. In some embodiments, the liquid inlet 302 is configured to receive liquid, and the liquid outlet 304 is configured to receive liquid flowing out of the liquid channel 306. In some embodiments, the liquid inlet 302 and the liquid outlet 304 are respectively, for example, tanks. The diameter of the liquid inlet 302 and the liquid outlet 304 ranges from 1.8 mm to 2.2 mm, and is, for example, 2 mm. The depth of the liquid inlet 302 and the liquid outlet 304 ranges from 1.8 mm to 2.2 mm, and is, for example, 2 mm.

The liquid channel 306 is, for example, a trench with a depth. In some embodiments, the liquid channel 306 has a liquid exchange region 306A located below the second hole part 220 of the oocyte incubation and fertilization system 200. The liquid exchange is performed between the liquid exchange region 306A and the oocyte incubation and fertilization system 200 through diffusion, that is, the liquid exchange is performed between the liquid perfusion system 300 and the oocyte incubation and fertilization system 200. In some embodiments, the liquid exchange region 306A has a contour such as a circle consistent with the second hole part 220.

In some embodiments, as shown in FIG. 1B, the integrated chip 10 includes, for example, a first film F1, a second film F2 below the first film F1, a third film F3 below the second film F2, and a fourth film F4 below the third film F3. In some embodiments, the material of the first film F1 to the fourth film F4 is, for example, PDMS, but the invention is not limited thereto. In some embodiments, the first film F1 includes a first through hole OP1 corresponding to the sperm inlet 102, a second through hole OP2 corresponding to the sperm outlet 104, a first trench T1 corresponding to the main channel 106 and the branch channel 108, a protrusion P corresponding to the obstruction structure 110, a third through hole OP3 corresponding to the tank 202 of the oocyte incubation and fertilization system 200, a fourth through hole OP4 corresponding to the liquid inlet 302, and a fifth through hole OP5 corresponding to the liquid outlet 304. The first through hole OP1 to the fifth through hole OP5 penetrate the first film F1, and the first trench T1 has a depth smaller than a thickness of the first film F1, for example.

The second film F2 includes a first hole part 210, a sixth through hole OP6 corresponding to the fourth through hole OP4, and a seventh through hole OP7 corresponding to the fifth through hole OP5. The second film F2 located below the first through hole OP1, the second through hole OP2, and the first trench T1 serves as the bottoms of the sperm inlet 102, the sperm outlet 104, and the main channel 106 and the branch channel 108, respectively. The third film F3 includes a second hole part 220, an eighth through hole OP8 corresponding to the sixth through hole OP6, and a ninth through hole OP9 corresponding to the seventh through hole OP7. The fourth film F4 includes a first groove CV1, a second trench T2, and a second groove CV2 connected to each other, the first groove CV1 corresponds to the eighth through hole OP8 and serves as the bottom of the liquid inlet 302, the second trench T2 serves as the liquid channel, the second groove CV2 corresponds to the ninth through hole OP9 and serves as the bottom of the liquid outlet 304. In other words, the fourth through hole OP4, the sixth through hole OP6, the eighth through hole OP8 and the first groove CV1 form the liquid inlet 302, and the fifth through hole OP5, the seventh through hole OP7, the ninth through hole OP9, and the second groove CV2 form the liquid outlet 304.

In some embodiments, the first film F1 to the fourth film F4 of the integrated chip 10 are manufactured, for example, by molding and drilling processes. The following description will be made by making the second film F2 including the first hole part as an example, but it is understood that the first film F1, the third film F3, and the fourth film F4 may also be produced by a similar method. First, a substrate (not shown) is provided, and columnar structures (not shown) arranged in an array is formed as a master model by a photolithographic process on the substrate. The columnar structures include a plurality of column patterns, and the height of the column pattern is, for example, the depth of the first hole, and the diameter of the column pattern is, for example, the diameter of the first hole. The substrate is, for example, a silicon wafer. The material of the columnar structures is, for example, a negative resist such as SU-8. Next, a material such as PDMS is applied to the surface of the substrate by spin coating or the like, and the material is filled in a gap between the columnar structures. After curing the material, the film including the first hole part is obtained by molding. Then, the sixth through hole OP6 and the seventh through hole OP7 are formed by the drilling process. Of course, in addition to the above method, other suitable methods such as etching may be used.

In some embodiments, for example, the adjacent two of the first film F1 to the fourth film F4 are adhered to each other by oxygen plasma modification. For example, the surfaces of the second film F2 to the fourth film F4 may be modified and adhered to the bottom surfaces of the first film F1 to the third film F3, respectively. In this way, the first film F1 to the fourth film F4 are combined to form the integrated chip 10. In some embodiments, for the convenience of carrying, the integrated chip 10 may be combined with a carrier board 20, and the material of the carrier board 20 may be glass. Furthermore, the integrated chip 10 may further include a sealing member (not shown) for controlling the pressure difference, so that the liquid temporarily stops flowing. For example, the sealing member may cover the sperm sorting system 100, and thus seal the sperm inlet 102 and the sperm outlet 104. Further, another sealing member may cover the oocyte incubation and fertilization system 200 to seal the tank 202. In some embodiments, the seal member may be formed of a material with a sealing property such as a tape.

Next, the method of sperm sorting, oocyte incubation, and in vitro fertilization through the integrated chip will be explained.

First, the integrated chip 10 is provided. Next, the sperm sorting system 100 is subjected to a wetting step to wet the main channel 106 and the branch channel 108. Specifically, the wetting solution is dropped into the sperm inlet 102 such that the solution flows through the main channel 106 and the branch channel 108 to wet them. In some embodiments, the wetting solution used for the wetting step is, for example, saline, germ cell culture medium or other liquid isotonic to sperms.

Next, the oocyte-containing sample is dropped into the tank 202 of the oocyte incubation and fertilization system 200 such that the oocytes are located in the first holes 212, respectively. In some embodiments, by controlling the dimension of the first hole 212, for example, only one oocyte is disposed in the first hole 212. Then, pre-treatment step is performed on the oocyte. In some embodiments, the pre-treatment step is, for example, an oocyte zona pellucida removal step, and performed by adding an enzyme to the tank 202. In some embodiments, after the pre-treatment step (for example, after the enzyme reacts completely), the step of opening the liquid perfusion system 300 is further included. That is, liquid B is supplied to the liquid inlet 302 such that the liquid B flows into the liquid outlet 304 through the liquid channel 306. The liquid B may be liquid used to exchange with the liquid in the tank 202, such as saline, germ cell culture medium or other liquid isotonic to sperms. In some embodiments, there is a pressure difference between the second hole part 220 and the liquid channel 306 (i.e., the liquid exchange region 306A) therebeneath, as a result, a harmful material such as an enzyme in the tank 202 may be discharged into the liquid channel 306 through the second hole part 220. In addition, the liquid B in the liquid channel 306 may also enter the tank 202 through the second hole part 220 for liquid exchange. In this way, the interior of the tank 202 of the oocyte incubation and fertilization system 200 may be maintained in an environment suitable for cultivating the oocyte or fertilized egg. Then, the tank 202 may be temporarily sealed by the sealing member. Herein, the sealing of the tank 202 may block the communication between the branch channel 108 and the tank 202 to prevent the material (such as an undesired sperm) from entering the tank 202 through the branch channel 108.

Next, the sperm-containing sample is dropped into the sperm inlet 102, so that the sperm sorting system 100 performs sperm sorting on the sperm-containing sample. In some embodiments, the sample may be a semen with human, pig, cow, or horse sperms. Then, after the sperm sorting step is completed, the oocyte incubation and fertilization system 200 is opened, so that the sorted high-efficiency sperm enters the tank 202 through the branch channel 108 to be fertilized with the oocyte to form a fertilized egg. Herein, the sorted high-efficiency sperm is a sperm that may be maintained near the branch channel 108, and the sorted high-efficiency sperm is, for example, a sperm with medium motility. In some embodiments, the sorting time is, for example, about 10 minutes, and the proportion of live sperm obtained by such sorting time is about 85%. In some embodiments, the method of opening the oocyte incubation and fertilization system 200 is, for example, to remove the sealing member. In addition, after the formation of the fertilized egg, the fertilized egg may be cultured in situ in the first hole 212. During the in-situ culture, the culture medium in the tank 202 may be replaced, specifically, by sucking the culture medium in the tank 202 and replenishing the fresh culture medium. In addition, generally, impuritie are included in the sperm-containing sample, and thus the filter component may be further disposed in the sperm inlet 102. Accordingly, the sperm-containing sample is filtered before entering the main channel 106 to avoid impuritie affecting subsequent sorting step and the like. In some embodiments, the filter component may be a filter film similar to the second hole part 220, for example, the material of the filter film may be PDMS, and the diameter and the depth of the hole of the filter film range from 25 μm to 35 μm and 50 μm to 100 μm, respectively.

Based on the above, the invention integrates the sperm sorting system, the oocyte incubation and fertilization system and the liquid perfusion system onto a single chip, in which high-efficiency sperm sorted by the sperm sorting system may be directly introduced into the oocyte incubation and fertilization system, and the high-efficiency sperm and the cultivated oocyte may be fertilized in situ to form a fertilized egg. Accordingly, in vitro fertilization is achieved. In particular, in the invention, sperm sorting, treatment on oocyte, and fertilized egg formation are passively performed by dropping the sperm containing sample into the sperm inlet and dropping the oocyte containing sample into the tank of the oocyte incubation and fertilization system without any other active processing. Passively performed sperm sorting, treatment on oocyte, and fertilized egg formation are gentle and harm to sperm and oocyte is avoid, and survival rate and fertilization rate are significantly increased. As a result, the chip of the invention may not only shorten the operation time of the traditional artificial reproduction technique, it also simplifies the operation process to avoid the death of the specimen due to the long operation time and the human-induced misoperation, thereby greatly improving the success rate of the artificial reproductive technique.

It will be apparent to those skilled in the art that various modifications and variations may be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. An integrated chip for sperm sorting, oocyte incubation, and in vitro fertilization, comprising:
a sperm sorting system, comprising a sperm inlet, a sperm outlet, a main channel, and a branch channel, wherein the main channel is connected to the sperm inlet and the sperm outlet, the main channel has a width which gradually increases along a direction from the sperm inlet toward the sperm outlet, and the branch channel is located between the sperm inlet and the sperm outlet and is connected to the main channel;
an oocyte incubation and fertilization system, connected to the branch channel, comprising a first hole part and a second hole part, wherein the first hole part comprises a plurality of first holes, a diameter of the first hole is greater than an average diameter of an oocyte for holding the oocyte to be fertilized by a sperm, the second hole part is located below the first hole part and comprises a plurality of second holes, a diameter of the second hole is smaller than the oocyte for liquid exchange, the sperm sorted by the sperm sorting system is introduced into the oocyte incubation and fertilization system through the branch charnel to fertilize with the oocyte located in the first hole; and
a liquid perfusion system, comprising a liquid inlet, a liquid outlet, and a liquid channel, wherein the liquid channel is located between and connected to the liquid inlet and the liquid outlet, a portion of the liquid channel is located below the second hole part, and liquid is exchanged between the oocyte incubation and fertilization system and the liquid channel through the second hole part.

2. The integrated chip of claim 1, a material of the first hole part and the second hole part comprises polydimethylsiloxane.

3. The integrated chip of claim 1, wherein the first holes are arranged regularly.

4. The integrated chip of claim 1, wherein the second holes are arranged regularly.

5. The integrated chip of claim 1, wherein the diameter of the second hole is greater than 30 um.

6. The integrated chip of claim 1, wherein the main channel comprises a first section and a second section connected to the first section, the first section is located between the sperm inlet and the second section, the second section is located between the first section and the sperm outlet, the first section has a substantially identical width, the second section has a gradually increasing width, and a minimum width of the second section is greater than the width of the first section.

7. The integrated chip of claim 6, wherein a width of the branch channel is smaller than the minimum width of the second section.

8. The integrated chip of claim 1, wherein the sperm sorting system further comprises an obstruction structure, the obstruction structure is located in the main channel and adjacent to the sperm outlet, and a distance is between the obstruction structure and a sidewall of the main channel.

9. The integrated chip of claim 8, wherein the obstruction structure is dumbbell-shaped.

10. The integrated chip of claim 1, wherein the sperm inlet and the sperm outlet are tanks respectively.

11. The integrated chip of claim 1, wherein the liquid channel has a liquid exchange region located below the oocyte incubation and fertilization system and having a contour consistent with the second hole part.

12. The integrated chip of claim 1, comprising:
a first film comprising a first through hole corresponding to the sperm inlet, a second through hole corresponding to the sperm outlet, a first trench corresponding to the main channel and the branch channel, a third through hole corresponding to the oocyte incubation and fertilization system, a fourth through hole corresponding to the liquid inlet, and a fifth through hole corresponding to the liquid outlet;
a second film located below the first film, comprising the first hole part, a sixth through hole corresponding to the fourth through hole, and a seventh through hole corresponding to the fifth through hole, wherein the second film located below the first through hole, the second through hole, and the first trench are respectively served as the sperm inlet, the sperm outlet, and bottoms of the main channel and the branch channel;
a third film located below the second film, comprising the second hole part, an eighth through hole corresponding to the sixth through hole, and a ninth through hole corresponding to the seventh through hole; and
a fourth film located below the third film, comprising a first groove, a second trench, and a second groove which are connected, wherein the first groove corresponds to the eighth through hole and serves as a bottom of the liquid inlet, the second trench serves as the liquid channel, the second groove corresponds to the ninth through hole and serves as a bottom of the liquid outlet.

13. The integrated chip of claim 12, wherein a material of the first film, the second film, the third film, and the fourth film comprises polydimethylsiloxane.

14. The integrated chip of claim 13, wherein the adjacent two of the first film, the second film, the third film, and the fourth film are adhered to each other.

15. A method for sperm sorting, oocyte incubation, and in vitro fertilization, comprising:
providing an integrated chip of claim 1;
dropping a sperm-containing sample into the sperm inlet, so that a sperm sorting is performed on the sperm-containing sample by the sperm sorting system;
dropping an oocyte-containing sample into the oocyte incubation and fertilization system, so that the oocytes is located in the first hole respectively; and
introducing the sorted sperm into the oocyte incubation and fertilization system through the branch channel, so that the sorted sperm and the oocyte are fertilized to form a fertilized egg in the first hole.

16. The method of claim 15, further comprising performing a zona pellucida removal step on the oocyte after the oocyte-containing sample is dropped into the oocyte incubation and fertilization system.

17. The method of claim 15, further comprising:
after the oocyte-containing sample is dropped into the oocyte incubation and fertilization system, sealing the oocyte incubation and fertilization system; and
after the sperm sorting of the sperm-containing sample is finished by the sperm sorting system, opening the oocyte incubation and fertilization system so that the sorted sperm enters the oocyte incubation and fertilization system through the branch channel.

18. The method of claim 15, further comprising:
after the sperm-containing sample is dropped into the sperm inlet, sealing the sperm sorting system; and
after the oocyte-containing sample is dropped into the oocyte incubation and fertilization system, opening the sperm sorting system to perform the sperm sorting.

19. The method of claim 15, further comprising culturing the fertilized egg in the oocyte incubation and fertilization system.

20. The method of claim 15, further comprising flowing liquid in the liquid perfusion system, wherein the liquid in the liquid perfusion system is exchanged with liquid in the oocyte incubation and fertilization system through the second hole part.
